(19) United States
(12) Patent Application Publication
(10) Pub. No.: US 2025/0295829 A1
(43) Pub. Date: Sep. 25, 2025

(54) PACKAGING FOR VOLATILE COMPOSITION DISPENSER CARTRIDGE

(71) Applicant: The Procter & Gamble Company, Cincinnati, OH (US)

(72) Inventors: Kumaresh GOVINDAN RADHAKRISHNAN, Singapore (SG); Joey Jia Yi LING, Singapore (SG); Mei San Gigi CHO, Singapore (SG)

(21) Appl. No.: 19/082,627

(22) Filed: Mar. 18, 2025

Related U.S. Application Data
(60) Provisional application No. 63/566,995, filed on Mar. 19, 2024.
(60) Provisional application No. 63/567,480, filed on Mar. 20, 2024.

(51) Int. Cl. A61L 9/12; B65D 81/107; B65D 83/00

(52) U.S. Cl. CPC A61L 9/127; B65D 81/107; B65D 83/771; A61L 2209/131; A61L 2209/133

(57) ABSTRACT

A consumer product is provided and includes an outer packaging, one or more cartridges packaged within the outer packaging, and a shock-absorbing insert within the outer packaging. The one or more cartridges include a reservoir containing a volatile composition in liquid form and having an opening, a membrane enclosing the opening of the reservoir, the membrane configured to allow evaporation of the volatile composition, a sealing substrate enclosing the opening of the reservoir, and a peripheral seal area in which a peripheral region of the reservoir and membrane are sealed together. The shock-absorbing insert supports the peripheral seal area of the one or more cartridges.

### FIELD OF THE INVENTION

The present disclosure relates to the field of packaging for cartridges used in volatile composition dispensers.

### BACKGROUND OF THE INVENTION

Continuous non-energised, membrane-based air freshening products are a consumer-friendly way to provide pleasant fragrances, and other air-enhancing products such as malodor reducing compounds, to an environment such as a room. Typically, these products are sold in the form of a cartridge comprising a perfume composition and a membrane through which the perfume composition may evaporate, where the cartridge is contained within a single-use plastic housing. An exemplary such product is that disclosed in WO2017/192638A1, which is a single-use product, resulting in plastic waste each time a product is disposed of.

In order to increase sustainability, it is desirable to provide such products with a refillable housing, into which a new cartridge may be placed once a previous cartridge has reached the end of its life. However, the provision of replacement cartridges involves difficulties ensuring that the cartridges are not damaged or activated during the supply chain. While existing products are typically provided within a rigid housing that protects the cartridge, a replacement cartridge would desirably be provided on its own to avoid excessive packaging waste. However, the absence of a rigid housing means that the cartridges are more likely to be damaged or activated during the supply chain.

Therefore, there is a need for a packaging for replacement cartridges that can prevent the cartridges from activating when subjected to knocks and vibrations during the supply chain.

### SUMMARY OF THE INVENTION

The current inventors have surprisingly found that a packaging as described herein advantageously reduces the incidence of damage and premature activation of cartridges when dropped or knocked during the supply chain.

Therefore, examples of the present invention provides the following.

1. A consumer product comprising:
(i) an outer packaging;
(ii) one or more cartridges packaged within the outer packaging, the one or more cartridges comprising:
   a reservoir containing a volatile composition in liquid form and having an opening;
   a membrane enclosing the opening of the reservoir, the membrane configured to allow evaporation of the volatile composition;
   a sealing substrate enclosing the opening of the reservoir; and
   a peripheral seal area in which a peripheral region of the reservoir and membrane are sealed together, and
(iii) a shock-absorbing insert within the outer packaging, wherein the shock-absorbing insert supports the peripheral seal area of the one or more cartridges.

2. The consumer product according to clause 1, wherein the shock-absorbing insert is in direct contact with the peripheral seal area of the one or more cartridges.

3. The consumer product according to clause 1 or 2, wherein the peripheral seal area comprises:
an outer peripheral seal area in which a peripheral region of the reservoir and membrane are sealed together; and
an inner peripheral seal area in which a peripheral region of the sealing substrate is sealed to the reservoir, wherein the shock-absorbing insert is configured to support the outer peripheral seal area and/or the inner peripheral seal area.

4. The consumer product according to clause 1 or 2, wherein:
the peripheral seal area comprises one or more secondary reservoir portions between the inner peripheral seal area and the outer peripheral seal area; and
the shock-absorbing insert is configured to support the one or more secondary reservoir portions.

5. The consumer product according to any one of the preceding clauses, wherein the shock-absorbing insert is substantially not in contact with a region of the one or more cartridges other than the peripheral seal area.

6. The consumer product according to any one of the preceding clauses, wherein at least 60% of a contact area between the shock-absorbing insert and the one or more cartridges is between the shock-absorbing insert and the peripheral seal area,
optionally at least 70%,
more optionally at least 80%, most optionally at least 90%.

7. The consumer product according to any one of the preceding clauses, wherein:
the one or more cartridges comprise a rupture mechanism located between the membrane and sealing substrate, the rupture mechanism configured to, upon actuation, rupture the sealing substrate; and
a projection of the rupture mechanism onto the membrane in a direction perpendicular to the membrane does not overlap with a contact area between the membrane and the shock-absorbing insert,
optionally wherein the rupture mechanism comprises one or more rupture elements configured to rupture the sealing substrate when an actuation pressure is applied to one or more actuation regions on the membrane, and the actuation regions do not overlap with a contact area between the membrane and the shock-absorbing insert.

8. The consumer product according to any one of the preceding clauses, wherein the outer packaging comprises a shock-absorbing portion located at a base region of the outer packaging,
optionally wherein one or more of (A) and (B) applies:
(A) the shock-absorbing portion increases the stability of the consumer product when placed on a flat surface, as measured by determining a minimum angle at which the consumer product topples;
(B) the shock-absorbing portion is formed from a paper-based material (e.g. paperboard) or a plastics material (e.g. polyethylene terephthalate).

9. The consumer product according to any one of the preceding clauses, wherein the shock-absorbing insert comprises:
a first part supporting the peripheral seal area of the one or more cartridges; and
a second part comprising one or more legs or flaps.

10. The consumer product according to any one of the preceding clauses, wherein the shock-absorbing insert comprises:
a sheet having one or more holes, where the reservoirs of the one or more cartridges are received by the one or more holes such that the peripheral seal areas of the one or more cartridges are supported by the sheet; and
one or more legs or flaps extending from the sheet,
optionally wherein the one or more legs or flaps are offset from a contact area between the sheet and peripheral seal areas.

11. The consumer product according to clause 9, wherein the shock-absorbing insert comprises:
a base portion;
a pair of intermediate walls extending from opposing edge regions of the base portion, the intermediate walls together comprising one or more pairs of opposing cut-out regions, where each pair of opposing cut-out regions forms a slot within which one of the one or more cartridges is disposed;
a pair of external walls extending from the base portion, the external walls being external to the intermediate walls; and
one or more legs or flaps extending from the base portion,
optionally wherein the one or more legs or flaps are offset from a contact area between the base portion and the one or more cartridges.

12. The consumer product according to any one of the preceding clauses, wherein the shock-absorbing insert comprises a paper-based material,
optionally wherein the shock-absorbing insert consists essentially of a paper-based material, more optionally wherein the shock-absorbing insert consists of a paper-based material.

13. The consumer product according to any one of the preceding clauses, wherein the shock-absorbing insert is formed from a single piece of paper-based material.

14. The consumer product according to any one of the preceding clauses, wherein the outer packaging does not contact a part of the one or more cartridges other than the peripheral seal area.

15. The consumer product according to any one of the preceding clauses, wherein the outer packaging does not contact the one or more cartridges.

16. The consumer product according to any one of the preceding clauses, wherein the outer packaging comprises a paper-based material and/or a plastics material,
optionally wherein the plastics material comprises polyethylene terephthalate (PET) and/or wherein the paper-based material comprises paperboard.

17. The consumer product according to any one of the preceding clauses, wherein the shock-absorbing insert is self-supporting when placed outside of the outer packaging.

18. The consumer product according to any one of the preceding clauses, wherein the consumer product comprises two or more cartridges and the two or more cartridges are not in direct contact.

### BRIEF DESCRIPTION OF THE FIGURES

FIG. 1 shows components of cartridges that may be present within the consumer product of the invention.

FIG. 2 shows a first view of a cartridge that may be present within the consumer product of the invention.

FIG. 3 shows a second view of a cartridge that may be present within the consumer product of the invention.

FIG. 4 shows a view of a cartridge that may be present within the consumer product of the invention.

FIGS. 5A and 5B show a shock-absorbing insert that may be used in the consumer product of the invention. FIG. 5A shows the shock-absorbing insert in its 3D form, while FIG. 5B shows a design of a 2D sheet that may be folded along fold lines to produce the structure shown in FIG. 5A.

FIG. 6 shows another type of shock-absorbing insert that may be used in the consumer product of the invention, and also shows how a cartridge may be inserted into the shock-absorbing insert.

FIG. 7 shows a design of a 2D sheet that may be folded along fold lines to produce the shock-absorbing insert shown in FIG. 6.

FIG. 8 shows the location of the fold lines shown in FIG. 7 when the shock-absorbing insert is folded.

FIG. 9 shows side views of consumer products according to a configuration, depicting the outer packaging.

FIG. 10 shows an outer packaging according to a configuration in flat form.

FIG. 11 shows a blister for a blister pack-type outer packaging.

FIG. 12 shows the order of impact locations for a drop test of shipper boxes containing the consumer products.

### DETAILED DESCRIPTION OF THE INVENTION

The inventors have surprisingly found that a consumer product comprising a shock-absorbing insert that supports volatile composition cartridges in the manner described herein advantageously reduces incidence of premature activation.

The invention, in an example, provides consumer product comprising:
(i) an outer packaging;
(ii) one or more cartridges packaged within the outer packaging, the one or more cartridges comprising:
   a reservoir containing a volatile composition in liquid form and having an opening;
   a membrane enclosing the opening of the reservoir, the membrane configured to allow evaporation of the volatile composition;
   a sealing substrate enclosing the opening of the reservoir; and
   a peripheral seal area in which a peripheral region of the reservoir and membrane are sealed together, and
(iii) a shock-absorbing insert within the outer packaging, wherein the shock-absorbing insert supports the peripheral seal area of the one or more cartridges.

### Cartridge

The invention provides a consumer product comprising a cartridge that may be used in a volatile composition dispenser.

The cartridge is typically a single-use disposable cartridge that contains a volatile composition for release to a surrounding environment, and once a cartridge is depleted of volatile composition it may be disposed of. The cartridge is typically for placing into a reusable housing, such that once the cartridge is depleted of volatile composition it may be removed from the housing and replaced by a new cartridge. The use of single-use cartridges with a reusable housing reduces the amount of material contributed to landfill as compared to products that are entirely single-use (i.e. where the housing is single-use), and also uses a lower volume of material (e.g. plastic) during the manufacturing process.

The cartridge comprises a reservoir, a membrane, and a sealing substrate. The reservoir contains the volatile composition and is enclosed by both the membrane and the sealing substrate, which may enclose the reservoir in any order. Thus, the membrane may enclose the sealing substrate, or the sealing substrate may enclose the membrane.

The cartridge comprises a peripheral seal area. The peripheral seal area may comprise an outer peripheral seal area, in which a peripheral portion of the reservoir and membrane may be sealed together. In configurations where the sealing substrate encloses the membrane, a peripheral portion of the sealing substrate may be sealed to a peripheral portion of the membrane at the outer peripheral seal area, i.e. a peripheral portion of each of the reservoir, membrane and sealing substrate may be sealed together at the outer peripheral seal area, provided that the sealing substrate may nevertheless be removed from the cartridge. This may be the case where the edges of the reservoir and membrane are coterminous. Alternatively, the edge of the reservoir may extend beyond the edge of the membrane, and the sealing substrate may be sealed directly to the reservoir at a peripheral region of the reservoir that is beyond the edge of the membrane. In configurations in which the membrane encloses the sealing substrate and the cartridge also comprises a rupture mechanism for rupturing the sealing substrate, the outer peripheral seal area may comprise only a peripheral portion of the reservoir and membrane. In such configurations, the peripheral seal area may also comprise an inner peripheral seal area in which an inner peripheral portion of the reservoir is sealed to the sealing substrate. This may be achieved by providing the reservoir with an intermediate step at an inner peripheral portion (e.g. between the outer peripheral portion and a main body of the reservoir), where the sealing substrate may be sealed to the intermediate step of the reservoir.

The reservoir, membrane, and sealing substrate are discussed in turn below.

### Reservoir

The reservoir may contain the volatile composition and has an opening that is enclosed by the membrane and sealing substrate.

The reservoir of the cartridge may typically be formed from a plastics material, which may advantageously be transparent to allow an easy view of a fill level of volatile composition within the reservoir. An example of a suitable material is polyethylene terephthalate (PET), which may preferably be obtained at least partially from recycled sources.

The reservoir may be configured for interfacing with a reusable housing. For example, the reservoir may be configured to be received by a window of a reusable housing, so that the cartridge is held securely within the housing when the housing is closed. The reservoir may therefore have a shape that is configured to correspond with a window of a reusable housing with which the cartridge is intended to be used, so the reservoir portion may be received and fit snugly within the window.

In such configurations, it may be advantageous for the reservoir to be formed from a transparent material, so that the fill level of volatile composition within the reservoir is visible from outside the reusable housing, such as through the window.

The cartridge may comprise one or more secondary reservoir portions. In some configurations, the secondary reservoir portions may be located at a peripheral seal area, such as between an inner peripheral seal area and an outer peripheral seal area described above. In such configurations, the volatile material may be present initially in the reservoir and be contained by the sealing substrate that is sealed to the reservoir at the inner peripheral seal area. Once the cartridge is activated, such as by rupturing of the sealing substrate, the volatile material may pass into the secondary reservoir portions and contact the membrane, which membrane is sealed to the reservoir at the outer peripheral seal area. When the cartridge comprises secondary reservoir portions as part of the peripheral seal area, the shock-absorbing insert may be configured to support the one or more secondary reservoir portions.

As mentioned above, the cartridge disclosed herein is typically a single-use cartridge for placing into a reusable housing. Thus, the cartridge typically does not comprise a housing of its own. In other words, the reservoir of the cartridge may be an outermost layer of the cartridge. In this context, “outermost” is to be understood as meaning that the cartridges do not include a substantial component outside the reservoir. For the avoidance of doubt, this does not exclude the presence of the membrane and sealing substrate enclosing an opening of the reservoir. In some configurations, the reservoir may nevertheless include a label or wrapping around the reservoir, which is intended to convey information to a user. However, the reservoir may be transparent as discussed herein, and in such cases the reservoir may typically not be covered by an additional label or wrapping so as to not obscure the reservoir.

### Membrane

The volatile composition is in liquid form and is configured to evaporate through a membrane. Accordingly, the cartridge comprises a microporous membrane, which for the sake of brevity may be referred to herein as “the membrane”. The membrane may enclose the reservoir such that volatile composition is unable to escape from the cartridge without passing through the membrane. The membrane may prevent the passage of liquid, such that the volatile composition is only able to escape the cartridge by evaporating through, or from, the membrane.

The membrane is vapor permeable and capable of wicking liquid, yet prevents free flow of liquid out of the membrane. Any suitable membrane may be used. Purely by way of example, certain properties that may result in advantageous membranes are discussed below. However, the invention is not limited to membranes having the properties below, and any membrane known in the art that allows the volatile composition to evaporate may be used in the invention.

The membrane may have any appropriate volume average pore diameter, such as from 0.01 μm to 0.5 μm, such as from 0.02 μm to 0.3 μm, such as from 0.05 μm to 0.2 μm, more particularly from 0.065 μm to 0.15 μm since this may provide improvements with regard to evaporation rate and controlling leakage or sweating of volatile composition. In certain configurations, the membrane may have a volume average pore diameter of from 0.065 μm to 0.15 μm, from 0.07 to 0.12 μm, from 0.07 to 0.11 μm, or 0.08 to 0.1 μm.

In some configurations, the membrane may have a pore size distribution such that at least 50%, such as at least 60%, such as at least 70%, such as at least 80% or such as at least 90% of the pores of the membrane have a pore diameter of from 0.065 μm to 0.15 μm.

The membrane may comprise (e.g. be formed from) any appropriate material, such as a polymer. A particular material that may be used is polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

The membrane may be formed from a single piece, or single sheet, of material. In other words, the membrane may be not laminated. Thus, the membrane may be formed from a single sheet of polyethylene having a thickness as described above.

Those of ordinary skill in the art will appreciate that the surface area of the membrane can vary depending on the user preferred size of the cartridge. In some configurations, the (evaporative) surface area of the membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

Particularly preferred membranes may have an evaporative surface area of from about 20 cm² to about 40 cm², such as from about 30 cm² to about 35 cm².

The membrane may form substantially all (e.g. at least 80%, at least 85%, at least 90% or at least 95%) of the surface area of a face of the cartridge. This advantageously allows the membrane to have a maximised evaporative surface area for the size of the cartridge, leading to improved release of volatile composition.

Thus, in some configurations the membrane may have an evaporative surface area of from about 20 cm² to about 40 cm², such as from about 30 cm² to about 35 cm² and form substantially an entire face of the cartridge.

In some configurations, the membrane may have an evaporative surface area of from about 20 cm² to about 40 cm², such as from about 30 cm² to about 35 cm² and the cartridge may have a maximum dimension of less than 10 cm, preferably less than 9 cm. This advantageously means that the cartridge has a compact size whilst retaining a high evaporative surface area. In such configurations, the membrane has a high size relative to the overall size of the cartridge, providing a high evaporation rate of volatile material.

The membrane may have any appropriate porosity. For example, the membrane may have a porosity of from 45% to 70%, on a volume basis, such as from 45% to 65%. In certain configurations, the porosity may be from 50 to 70%, such as 55 to 65%.

The membrane may have any appropriate total pore volume, such as from 0.6 to 2 cm³/g. Typically, the total pore volume may be from 0.65 to 1.6 cm³/g, such as 0.7 to 1.5 cm³/g. In certain configurations, the total pore volume may be from 0.8 to 1.4 cm³/g.

The membrane may have any appropriate bulk density, such as from 0.3 to 0.8 g/cm³. Typically, the bulk density may be from 0.35 to 0.75 g/cm³, such as from 0.4 to 0.7 g/cm³. In certain configurations, the bulk density may be from 0.4 to 0.6 g/cm³.

Suitable membranes for the present invention include polyethylene membranes having the properties described herein, available from Microporous, LLC.

The membrane may comprise any suitable filler and plasticizer known in the art. Fillers may include finely divided silica, clays, zeolites, carbonates, and mixtures thereof. In one configuration, the membrane may be filled with about 30% to about 80%, by total weight, of silica.

In one aspect of the invention, the membrane may include a dye that is sensitive to the amount of volatile composition it is in contact with to indicate end-of-life. Alternatively, the membrane may change to transparent when in contact with a fragrance or volatile composition to indicate diffusion is occurring. Other means for indicating end-of-life that are known in the art are contemplated for the present invention.

### Sealing Substrate

The cartridge comprises a sealing substrate that encloses the reservoir, and hence, encloses the volatile composition. This prevents evaporation of the volatile composition for as long as the sealing substrate is in place and intact. The sealing substrate may be removed or ruptured to allow the volatile composition to evaporate. This removal or rupturing may be referred to herein as “activating” or “activation of” the cartridge. The cartridge is configured to be activated before use, i.e. the sealing substrate is configured to be ruptured or removed before use. In this context, “use” refers to enabling the volatile composition to evaporate from the cartridge, i.e. perform its function of dispensing the volatile composition by evaporation. In other words, rupturing or removing the sealing substrate enables evaporation of the at least one liquid volatile composition from the cartridge.

Non-limiting examples of suitable sealing substrates include an impermeable film, foil, or laminate, such as a flexible (e.g. polymeric) film, a flexible (e.g. metal) foil, or a composite material (e.g. a foil/polymeric film laminate). The impermeable film, foil or laminate is provided adhered to the cartridge to prevent evaporation of volatile composition. A particular example of a suitable sealing substrate is aluminium foil.

The sealing substrate may have any appropriate thickness, such as from 10 μm to 1 mm, from 15 μm to 100 μm, from 18 μm to 50 μm, or from 20 μm to 35 μm.

As mentioned above, there are two possible configurations for the order of the sealing substrate and the membrane.

In some configurations, the sealing substrate may be positioned between the membrane and the volatile composition, preventing the volatile composition from contacting the membrane. In such configurations, the cartridge may comprise a rupture mechanism. Thus, the cartridge may be configured for use with a housing that comprises rib elements that, upon closure of the housing, actuate the rupture mechanism, causing rupture of the sealing substrate and allowing volatile composition to pass through the ruptured sealing substrate and come into contact with the membrane. The volatile composition may then impregnate the membrane, from which it may evaporate. Suitable rupture mechanisms are described in detail in U.S. Pat. Nos. 10,561,754, 10,561,755 and 10,561,756.

However, a problem with rupture mechanisms of this type is that they may activate prematurely if the cartridges are dropped or subjected to shocks, such as during transport in a supply chain. This premature activation is alleviated by the packaging described herein, which reduces the transmission of shocks and vibrations to the cartridges.

In other configurations, the membrane may be positioned between the volatile composition and the sealing substrate, such that the volatile composition is in contact with the membrane but cannot evaporate and leave the cartridge because the membrane is enclosed by the sealing substrate. In such configurations, the cartridge may also be configured for use with a housing that, like the foregoing, comprises ribs which directly rupture the sealing substrate. However, this may result in only small holes in the sealing substrate, limiting the rate at which the volatile composition may evaporate from the cartridge. Therefore, in such configurations, the sealing substrate may be provided as a removable component (such as a removable film, foil or laminate) that encloses the membrane and prevents evaporation of the volatile composition before the sealing substrate is removed. When a sealing substrate of this type is removed from the cartridge, the entire membrane may be exposed to a surrounding environment, enabling an increased evaporation rate of volatile composition. In such cases, the sealing substrate may be provided with a pull tab to allow for easy removal of the sealing substrate from cartridge prior to use by a consumer.

However, a problem with removable sealing substrates of this type is that they may suffer from delamination, especially when subjected to repeated shocks or vibrations, such as during transport in a supply chain. This delamination is alleviated by the packaging described herein, which reduces the transmission of shocks and vibrations to the cartridges.

As described, in some configurations, the sealing substrate may be a rupturable substrate. In other configurations, the sealing substrate may be a removable sealing substrate.

### Shock-Absorbing Insert

The shock-absorbing insert serves to absorb shocks and vibrations as they pass through the consumer product. When a force is applied to the consumer product, such as a force caused by the consumer product being dropped and impacting the ground, the shock-absorbing insert absorbs some of the force, thereby reducing the force that is ultimately transferred to the cartridge. This reduces the chance of damaging or prematurely activating the cartridge.

The shock-absorbing insert supports the peripheral seal area of the one or more cartridges. Since the shock-absorbing insert contacts the peripheral seal area of the one or more cartridges, forces transferred from the shock-absorbing insert to the one or more cartridges are transferred to the peripheral seal area. Forces transferred to this region of the cartridge may be less likely to cause premature activation of the cartridge, especially when the cartridges comprise a rupture mechanism located underneath the membrane, which rupture mechanism is configured to be activated by applying pressure at specific areas of the membrane. In contrast, when a force is applied to the membrane, it may cause unwanted activation of a rupture mechanism.

Therefore, in some configurations, the shock-absorbing insert is in direct contact with the peripheral seal area of the one or more cartridges. In some configurations, the shock-absorbing insert is substantially not in contact with a region of the one or more cartridges other than the peripheral seal area. This reduces the possibility of forces being transferred directly to vulnerable areas of the cartridge, such as the membrane or a rupture mechanism. In this context, “substantially not in contact with” may refer to the majority of a contact area between the cartridge and shock-absorbing insert being between the peripheral seal area and the shock-absorbing insert. For example, at least 60%, at least 70%, at least 80%, or at least 90% of a contact area between the cartridge and shock-absorbing insert may be between the peripheral seal area and the shock-absorbing insert. For the avoidance of doubt, where the shock-absorbing insert is substantially not in contact with a region of the one or more cartridges other than the peripheral seal area, this does not exclude the possibility of a small part of the shock-absorbing insert (e.g. an edge of a sheet) being in contact with other parts of the cartridge. For example, in some configurations an edge part of the shock-absorbing insert may be in contact with the reservoir of the cartridges.

When the cartridge comprises a rupture mechanism as described herein, it may be advantageous for a projection of the rupture mechanism onto the membrane in a direction perpendicular to the membrane to not overlap with a contact area between the membrane and the shock-absorbing insert. This reduces the chance of forces being transferred from the shock-absorbing insert to the rupture mechanism, reducing the chance of premature activation. In particular, where the cartridge comprises a rupture mechanism, the rupture mechanism may comprise one or more components (e.g. rupture elements) that are actuated when a sufficient pressure (“actuation pressure”) is applied to one or more regions on the membrane (“actuation regions”), where such actuation ruptures the sealing substrate. In these configurations, a contact area between the membrane and shock-absorbing insert preferably does not overlap with the actuation regions.

The shock-absorbing insert may comprise a first part that supports (e.g. is configured to contact) the peripheral seal area of the one or more cartridges. The shock-absorbing insert may also comprise a second part comprising one or more legs or flaps. Typically, the legs or flaps primarily serve to absorb shocks, though the entire shock-absorbing insert may be capable of absorbing shocks. For example, the legs or flaps may be configured to deform, flex or bend when subjected to a force, thereby absorbing some of the force and reducing the force transferred to the cartridges. Any suitable number of legs or flaps may be present. A basic shock-absorbing insert may comprise a single flap at one end, with the remaining portion of the shock-absorbing insert forming the first part that supports the peripheral seal areas. A shock-absorbing insert of this type may have the appearance of a ramp, with a longer first part and a shorter flap. Other examples of suitable shock-absorbing inserts include those with multiple legs or flaps surrounding and reinforcing a flat first part, in a table-like arrangement. A skilled person will appreciate that any suitable number of legs or flaps may be present.

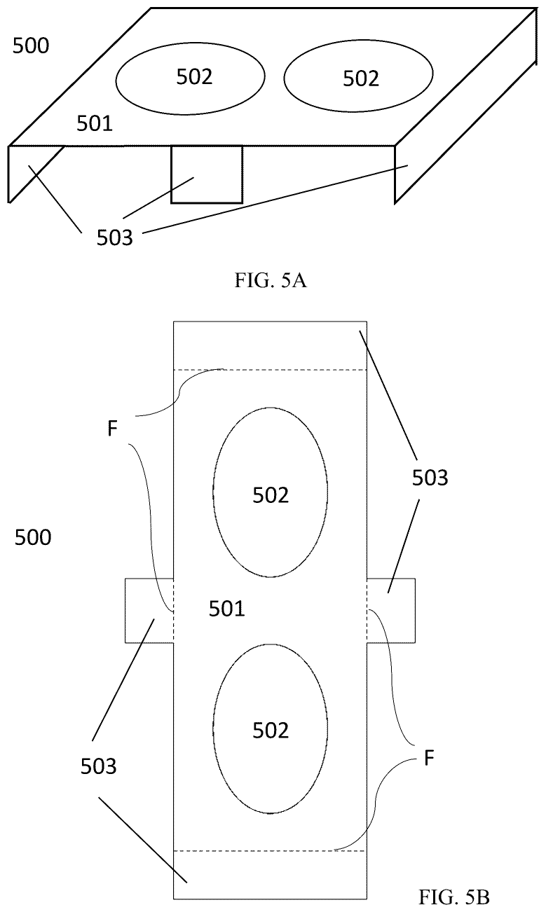

In some configurations, the shock-absorbing insert may comprise a sheet having one or more holes (e.g. two or more, such as two), where the reservoirs of the one or more cartridges are configured to be received by the one or more holes, such that the peripheral seal areas of the one or more cartridges are supported by the sheet. The shock-absorbing insert may also comprise one or more (e.g. 2 to 8, such as 2 to 6, such as 4) legs or flaps extending from the sheet. The legs or flaps may be offset (e.g. laterally separated in the plane of the sheet) from a contact area between the sheet and peripheral seal areas, to reduce force transfer from the legs or flaps to the cartridges. As a force is transferred from the legs or flaps to the sheet, it must then travel along the sheet before reaching the peripheral seal areas, and the sheet is able to absorb some of the force before it is transferred to the cartridge. An example of this configuration is shown in FIGS. 5A and 5B discussed hereinbelow.

In some configurations, the shock-absorbing insert may comprise:
a base portion;
a pair of intermediate walls extending from opposing edge regions of the base portion, the intermediate walls together comprising one or more pairs of opposing cut-out regions, where each pair of opposing cut-out regions forms a slot within which one of the one or more cartridges is disposed;
a pair of external walls extending from the base portion, the external walls being external to the intermediate walls; and
one or more legs or flaps extending from the base portion.

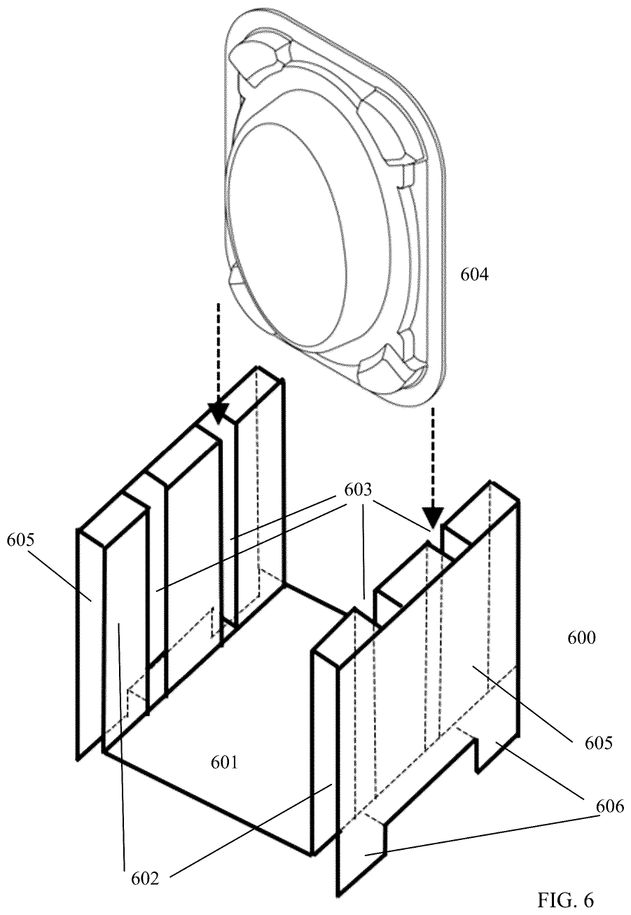

In this way, the cartridges may be held within slots in the shock-absorbing insert. The base may is present at one end of the slots, such that the base may support an edge part of the cartridges, such as an edge of the peripheral seal area. The other end of the slots is open, such that the cartridges may be slid into the slots, and the intermediate walls support the peripheral seal areas of the cartridges. The external walls may support edge parts of the peripheral seal area, much like the base. The shock-absorbing insert also comprises legs or flaps extending from the base portion in an opposing direction to the intermediate and external walls. Typically, the one or more legs or flaps may be offset (e.g. laterally separated in the plane of the base) from a contact area between the base portion and the one or more cartridges. This reduces force transfer from the legs or flaps to the cartridges. An example of this configuration is shown in FIG. 6 discussed hereinbelow.

The shock-absorbing insert may be formed from any appropriate material. Typically, the shock-absorbing insert is formed from a material that is able to bend or deform in response when subjected to a force, to absorb the force. Typically, the shock-absorbing insert is able to at least partially revert to its original state after being subjected to a force, so that it is able to absorb subsequent shocks. In some configurations, the shock-absorbing insert may be formed from a paper-based material. This provides environmental advantages, and also allows for a simple manufacturing process.

In some configurations, the shock-absorbing insert may be formed from a single piece of material. For example, the shock-absorbing insert may be formed from a single sheet that may be folded and/or cut to form the shock-absorbing insert. In some configurations, the shock-absorbing insert may comprise a single piece of paper-based material. In further configurations, the shock-absorbing insert may consist essentially of, or consist of, a paper-based material. For example, the shock-absorbing insert may comprise at least 60%, at least 70%, at least 80%, at least 90%, at least 95%, at least 97% or at least 99% of a paper-based material. In some configurations, the shock-absorbing insert may comprise at least 90% of a paper-based material.

Any appropriate paper-based material may be used, such as paper, paperboard or cardboard. In some configurations the paper-based material comprises paperboard. In some configurations the paper-based material comprises cardboard. The paper-based material may be laminated with a plastics material. In some configurations the paper-based material is not laminated with a plastics material.

In some configurations, the consumer product comprises two or more cartridges. In order to minimise force transmission from one cartridge to another, it is preferable for the two or more cartridges to not be in direct contact. This may be achieved by designing the shock-absorbing insert to ensure distance between each cartridge in the consumer product.

The shock-absorbing insert may be self-supporting, e.g. able to stand up on its own, when placed outside of the outer packaging. This advantageously allows a consumer to remove the shock-absorbing insert from the outer packaging and use it to store the cartridges in a more compact manner, such as in a small cupboard. By storing the cartridges in the shock-absorbing insert, they may be held safely and securely, reducing the chance of them being dropped and activating prematurely. In some configurations, the shock-absorbing insert may have a toppling angle of at least 15°, which may be determined as a minimum toppling angle in four orthogonal directions, e.g. at 0°, 90°, 180° and 270°.

For the avoidance of doubt, the consumer product may comprise multiple shock-absorbing inserts. For example, multiple shock-absorbing inserts may be provided to support a single cartridge, e.g. two shock-absorbing inserts, each supporting a different side of the cartridge. Alternatively, multiple shock-absorbing inserts may be provided where each shock-absorbing inserts supports only one or some of the cartridges present in the consumer product.

### Outer Packaging

The outer packaging may be any appropriate packaging, such as a rigid, semi-rigid or soft packaging. From the perspective of protecting the cartridges against accidental damage or rupture, the packaging may typically be rigid or semi-rigid. It may be preferable to construct the outer packaging from semi-rigid materials, since the ability to deform in response to applied forces will absorb shocks and vibrations, reducing the degree to which they are transferred to the contents inside the outer packaging. For example, the packaging may be formed from a plastics material, a paper-based material, or a combination thereof.

In some configurations, the outer packaging may be formed from a majority of semi-rigid materials, such as a combination of plastics materials and paper-based materials, or a majority of paper-based materials. Paper-based materials may absorb more shocks than plastics materials, helping to reduce forces transferred to the cartridges. Thus, the outer packaging may be formed from at least 50 wt. % paper-based materials, such as at least 60 wt. %, at least 70 wt. %, at least 80 wt. %, at least 90 wt. %, at least 95 wt. % paper-based materials.

The outer packaging may take any appropriate form. In some configurations, the outer packaging may take the form of a blister or clam-shell packaging that may be made from a plastics material, a paper-based material, or a combination thereof. A blister packaging may have any appropriate construction, including a face seal blister, full-face seal blister, full card blister, and trapped blister. In some configurations, the outer packaging may take the form of a box, such as a paper-based box.

The consumer product may also comprise one or more shock-absorbing portions at a base region of the outer packaging. The shock-absorbing portions absorb shocks applied to the base of the outer packaging, such as when the outer packaging is dropped, or when the outer packaging is vigorously placed onto a hard surface such as a table. The shock-absorbing portion may be made from any appropriate material, such as a deformable plastics material or a paper-based material. In some configurations, the shock-absorbing portion may comprise a paper-based material. In further configurations, the shock-absorbing portion may consist essentially of, or consist of, a paper-based material. For example, the shock-absorbing portion may comprise at least 60%, at least 70%, at least 80%, at least 90%, at least 95%, at least 97% or at least 99% of a paper-based material. In some configurations, the shock-absorbing portion may comprise at least 90% of a paper-based material.

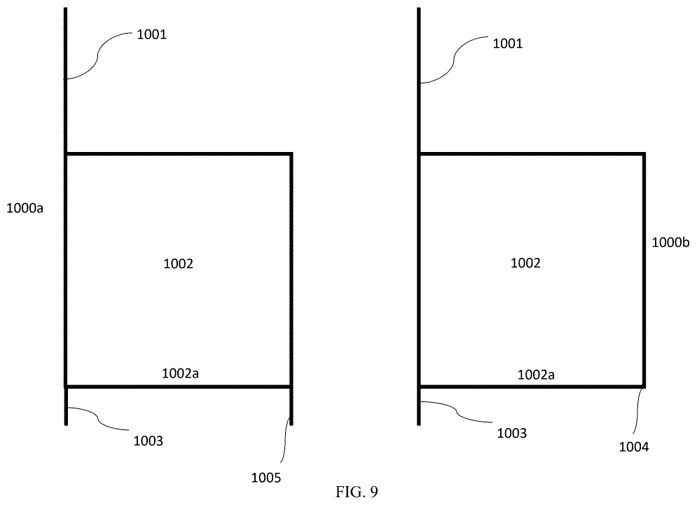

As used herein, the term “shock-absorbing portion” is to be understood as a portion of the outer packaging that prevents contact between a base region of the outer packaging and a flat surface, when the consumer product is dropped with its base region facing downwards. Thus, the presence of a single protrusion or bulge extending from the base region may not function as a shock-absorbing portion because when such a consumer product is dropped, the protrusion/bulge will impact the ground first, but the consumer product will subsequently tip to have a second point of contact with the ground. If this second point of contact is not a shock-absorbing portion then shocks will be transferred into the consumer product, and may cause premature activation of cartridges contained therein. This may be understood with reference to FIG. 9 below.

The shock-absorbing portion may also stabilise the outer packaging when placed on a flat surface, reducing the chance of the consumer product toppling or falling over, which can cause activation of cartridges within the consumer product, especially when toppling on a hard surface. The presence of shock-absorbing portion may also help the consumer product land in an upright position when dropped, rather than landing and then subsequently falling over, which exposes the cartridges to two individual shocks: one from the initial drop and a second from the subsequent falling over.

Therefore, in some configurations, the shock-absorbing portion provides a stable base. In some configurations, the shock-absorbing portion increases the stability of the consumer product when placed on a flat surface, as measured by determining a toppling angle at which the consumer product topples and comparing this against a toppling angle at which an equivalent consumer product not having the shock-absorbing portion(s) topples. The toppling angle may be determined as a minimum toppling angle measured in four orthogonal directions, e.g. at 0°, 90°, 180° and 270.

In some configurations, the outer packaging may comprise a plurality of shock-absorbing portions to provide a stable base. For example, the outer packaging may comprise two elongated shock-absorbing portions at two opposing sides of the outer packaging, or the outer packaging may comprise a plurality of shock-absorbing portions at corners of the outer packaging.

When the shock-absorbing portion is formed from a plastics material, it may take the form of a bulge or protrusion extending from the outer packaging. This bulge or protrusion may be configured to slightly crumple, flex or otherwise deform when subjected to a force, such as due to being dropped. The crumpling of the shock-absorbing portion absorbs force and reduces the force transferred to the cartridges. However, it is desirable that the plastics material does not deform too severely, as this may give the appearance of a damaged product, which is undesirable to consumers and may cause them not to purchase the consumer product. Therefore, it may be desirable for the shock-absorbing portion to be formed from polyethylene terephthalate (PET), and have a thickness that allows it to deform when dropped, such as about 0.3 mm to about 0.7 mm, e.g. about 0.5 mm.

When the shock-absorbing portion is formed from a paper-based material, it may take the form of one or more (e.g. two or more) legs or flaps that may be configured to deform, flex or bend when subjected to a force, thereby absorbing some of the force and reducing the force transferred to the cartridges.

Suitable paper-based materials that may be utilized in the outer packaging include paperboard and cardboard (such as single face, single wall, double wall, and triple wall cardboard). In some configurations, the outer packaging may comprise paperboard. In some configurations, the paper-based materials may be laminated with a plastics material.

Suitable plastics materials that may be utilized in the outer packaging include any transparent plastics material that is able to adequately protect the cartridges. Specific examples include PET (polyethylene terephthalate) and PVC (polyvinyl chloride), though a skilled person will appreciate that other plastics may be used. In some configurations, these materials may be obtained from recycled sources. The plastics materials may have any appropriate thickness, such as from about 0.2 mm to about 1 mm, e.g. about 0.3 mm to about 0.7 mm, such as about 0.5 mm.

In some configurations, the outer packaging does not contact a part of the one or more cartridges other than the peripheral seal area. This reduces force transfer from the outer packaging to delicate parts of the cartridges, such as the membrane and when present, rupture mechanism. In some configurations, the outer packaging does not contact the one or more cartridges.

Various configurations will now be described to provide an overall understanding of the principles of the structure, function, manufacture, and use of the products disclosed herein. One or more examples of these configurations are illustrated in the accompanying Figures. Those of ordinary skill in the art will understand that the products specifically described herein and illustrated in the accompanying Figures are non-limiting example configurations and that the scope of the various configurations of the present disclosure are defined solely by the claims. The features illustrated or described in connection with one example configuration may be combined with the features of other example configurations. Such modifications and variations are intended to be included within the scope of the present disclosure.

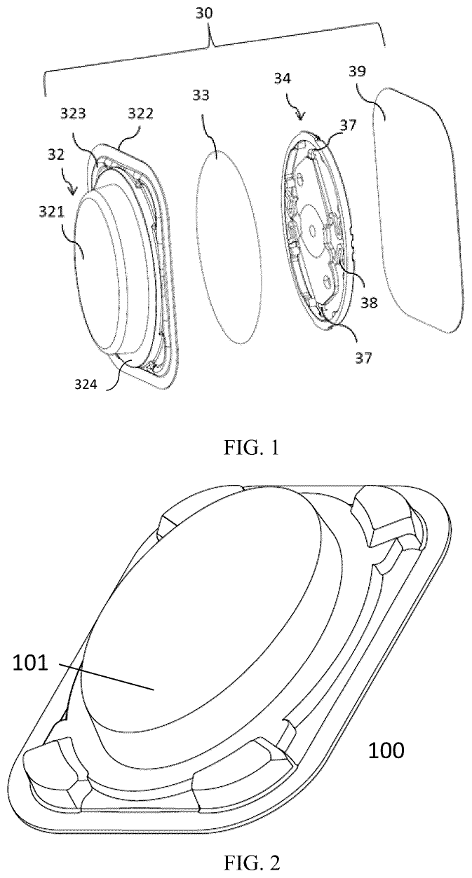

FIG. 1 shows an expanded view of a cartridge 30 according to a configuration. The cartridge 30 comprises a reservoir 32 having a main reservoir body 321, within which the volatile composition is stored. A sealing substrate 33 is sealably attached to and encloses the reservoir 32 at an inner peripheral seal area 324 to prevent the volatile composition from being released until the cartridge 30 is activated. The sealing substrate 33 may be ruptured to release the volatile composition by actuating a rupture mechanism 34 positioned adjacent to the sealing substrate 33. One or more rupture elements 37 are arranged within the rupture mechanism 34 to puncture holes in the sealing substrate 33. The rupture element 37 may be a pin. The cartridge 30 may comprise a membrane 39 located on the exterior of the cartridge 30. The membrane 39 may be sealably attached to an outer peripheral seal area 322 located at a peripheral seal area 323 of the reservoir 32. The peripheral seal area 323 also comprises the inner peripheral seal area 324 in which the sealing substrate 33 is sealably attached to the reservoir 32. The membrane 39 may enclose the reservoir 32, the volatile composition, the sealing substrate 33, and the rupture mechanism 34. The membrane 39 may be configured to flex when a pressure or an actuation force is applied on the membrane 39. And the membrane 39 may be configured to porous so as to sustainably absorb and volatilize the volatile composition.

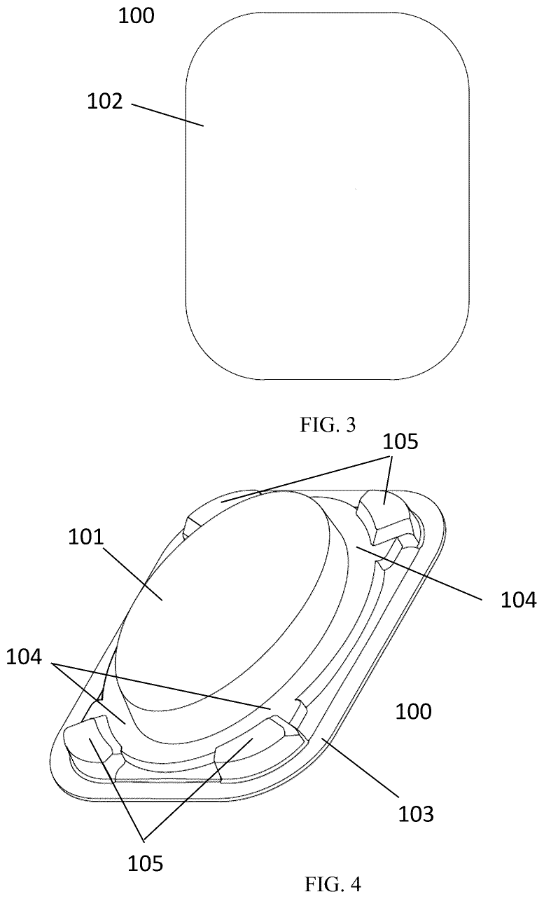

FIGS. 2-4 shows a cartridge 100 according to a different configuration, comprising a reservoir 101 containing a volatile composition. Although not shown in FIG. 2, the cartridge 100 comprises a membrane 102 on the reverse side, as shown in FIG. 3. The membrane 102 encloses the opening of the reservoir 101 and is configured to allow evaporation of the volatile composition. The cartridge 100 comprises a sealing substrate (not shown) enclosing the opening of the reservoir; and has a peripheral seal area in which a peripheral region of the reservoir and membrane are sealed together.

The parts of the peripheral seal area are shown in more detail in FIG. 4. The peripheral seal area is composed of an outer peripheral seal area 103 in which a peripheral region of the reservoir and membrane are sealed together, an inner peripheral seal area 104 in which a peripheral region of the sealing substrate (not shown) is sealed to an inner peripheral region of the reservoir, and secondary reservoir portions 105 that are between the outer and inner peripheral seal areas.

FIG. 5A shows a shock-absorbing insert 500 comprising a sheet 501 having two holes 502, where the reservoir of cartridges (not shown) may be received by the holes 502 such that the peripheral seal area of the cartridges are supported by the sheet 501. The shock-absorbing insert 500 also comprises legs 503 in the form of flaps extending from the sheet. The flaps may bend in response to shocks, absorbing energy and limiting the transfer of energy into a cartridge that is supported in the shock-absorbing insert 500. The legs 503 are offset from a contact area between the sheet and peripheral seal areas, which contact area is immediately surrounding the hole 502. This helps to minimise energy transfer from the legs 503 into a cartridge, since some of the energy will be absorbed by the sheet 501 as it passes through the shock-absorbing insert 500. FIG. 5B shows the shock-absorbing insert 500 of FIG. 5A in flat form. The shock-absorbing insert 500 of FIG. 5A may be obtained by folding along the fold lines F.

FIG. 6 shows a shock-absorbing insert 600 comprising a base portion 601 and a pair of intermediate walls 602 extending from opposing edge regions of the base portion 601. The intermediate walls 602 together comprise pairs of opposing cut-out regions 603, where each pair of opposing cut-out regions 603 forms a slot within which a cartridges 604 is to be disposed. The arrows show how the cartridge 604 may be slid into the slot. The shock-absorbing insert 600 also comprises a pair of external walls 605 extending from the base portion 601, the external walls 605 being external to the intermediate walls 602. Legs 606 extend from the base portion, and in this configuration are present as an extension of the external wall 605. The legs serve to absorb shocks and limit the energy transfer into a cartridge 604 held within the shock-absorbing insert 600. This effect is improved by placing the legs in a position that is offset from a contact area between the base portion 601 and the cartridge. While the shock-absorbing insert 600 is depicted with two pairs of cut-out regions 603, a skilled person will appreciate that alternative shock-absorbing inserts may be provided with other numbers of cut-out regions.

The dashed lines in FIG. 6 depict hidden edges or fold lines, and FIG. 8 shows the same shock-absorbing insert as FIG. 6 but with the dashed lines removed for clarity.

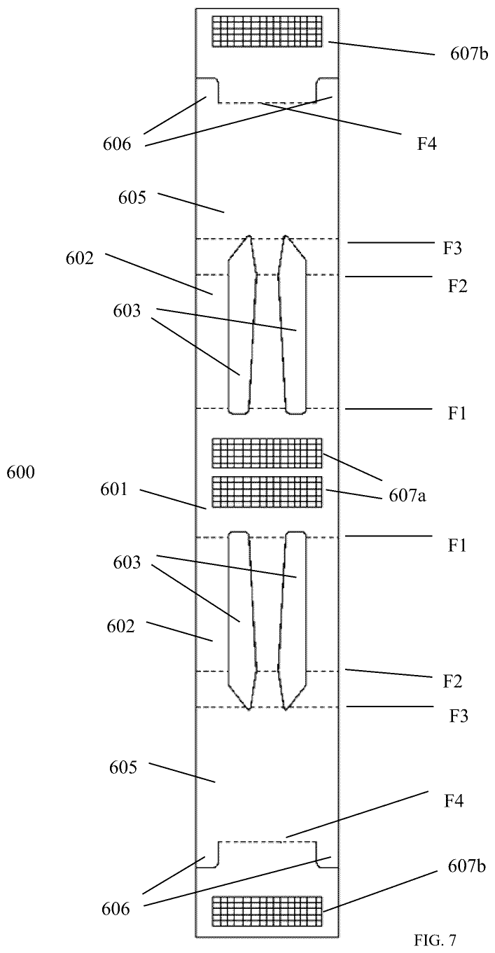

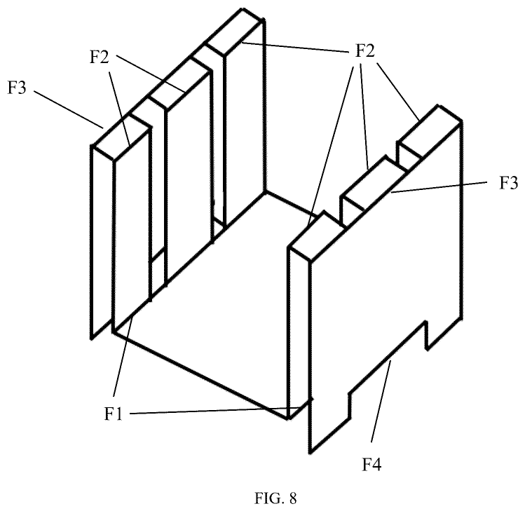

The shock-absorbing insert of FIG. 6 may be made from a single piece of paper-based material, such as paperboard, as shown in FIG. 7. The shock-absorbing insert 600 may be obtained by folding along each of the dashed fold lines F1-F4 and adhering the adhesive portions 607a and 607b together. The adhesive portions 607a are located on the underside of the base portion 601, and the adhesive portions 607b are folded under the base portion and adhered to the adhesive 607a. This fold also forms the legs 606. The location of the fold lines F1-F4 is shown in the context of the 3D product in FIG. 8.

FIG. 9 shows side views of two examples of consumer products having outer packagings 1000a and 1000b, which may be, for example, blister packagings with a paper-based backing portion, or may be entirely paper-based packagings. The packagings 1000a and 1000b have a backing portion 1001, which may comprise a hole from which the packagings 1000a and 1000b may be hung, e.g. in a retail store. The packagings comprise a storage region 1002 within which the cartridges, shock-absorbing insert, and if desired a housing configured to receive the cartridges, may be placed. The storage region 1002 has a base 1002a which forms the base region of the consumer product. The backing portion 1001 includes a part that extends below the storage region 1002, as is common for consumer products having this type of packaging (whether a blister packaging or an entirely paper-based packaging). However, when the packaging 1000b is dropped, this part 1003 will impact the ground first, and after this impact the packaging will tip and the front bottom edge 1004 of the storage region 1002 will impact the ground, transferring a shock to the contents of the storage region 1002. Accordingly, the part 1003 cannot solely function as a shock-absorbing portion. The packaging 1000a comprises a shock-absorbing portion 1005, which will impact the ground at a similar time to part 1003, absorbing shocks and reducing the forces transferred to the contents of the storage region 1002.

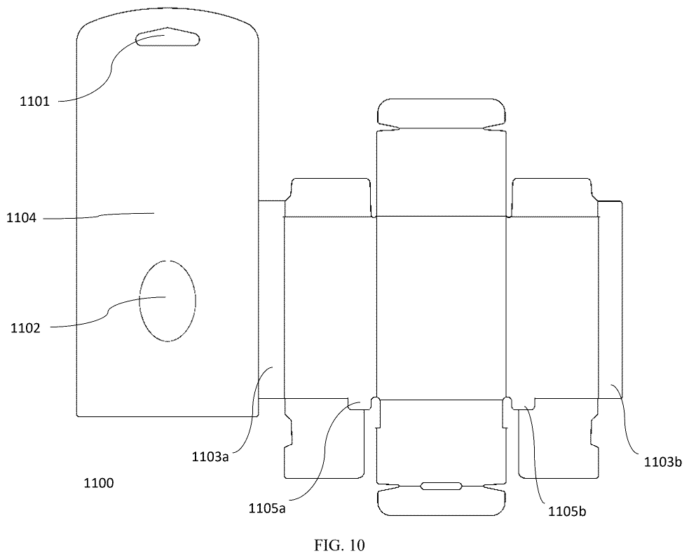

FIG. 10 shows an outer packaging 1100 in flat form. The outer packaging 1100 may be folded to provide a 3D outer packaging, a side view of which is shown in FIG. 9. The outer packaging 1100 may comprise a first hole 1101 for hanging the consumer product, and may comprise a second hole 1102 for viewing cartridges held within the outer packaging 1100. The outer packaging 1100 may be folded to a 3D form by adhering flaps 1103a and 1103b to the backing portion 1104, and folding the remaining portions together. The outer packaging 1100 comprises shock-absorbing portions 1105a and 1105b. Typically, an outer packaging 1100 of the type shown in FIG. 10 may comprise a shock-absorbing insert of the type shown in FIGS. 6-8. The outer packaging 1100 may be formed from any appropriate material, but is especially suitable for paper-based materials.

FIG. 11 shows a blister 1200 for use in a blister pack-type consumer product. The blister 1200 may be attached to a backing portion, such as a paper-based backing portion (not shown) to form an outer packaging. The blister 1200 comprises a first section 1201 for containing a housing into which cartridges may be placed, and a second section 1202 for containing two cartridges supported by a shock-absorbing insert. The blister 1200 may typically contain a shock-absorbing insert of the type shown in FIGS. 5A and 5B. Thus, the legs/flaps of the shock-absorbing insert may contact the backing portion (not shown), with the sheet of the shock-absorbing insert contacting a first shoulder portion 1203 of the blister. The cartridges may be held securely by the blister, with a second shoulder portion 1204 in contact with the peripheral seal area (e.g. an outer peripheral seal area) of the cartridges. The second section 1202 provides a space between the outside of the blister and the membrane of the cartridges, to protect the cartridges from external impact that causes deformation of the blister. The blister 1200 also comprises a shock-absorbing portion 1205, which may slightly deform when the blister 1200 is dropped, thereby absorbing shocks and limiting the transfer of force to cartridges contained within. The shock-absorbing portion 1205 also helps provide a stable base, when combined with the first section 1201, allowing the blister to stand upright on a flat surface. The blister 1200 may be formed from any appropriate material, such as polyethylene terephthalate.

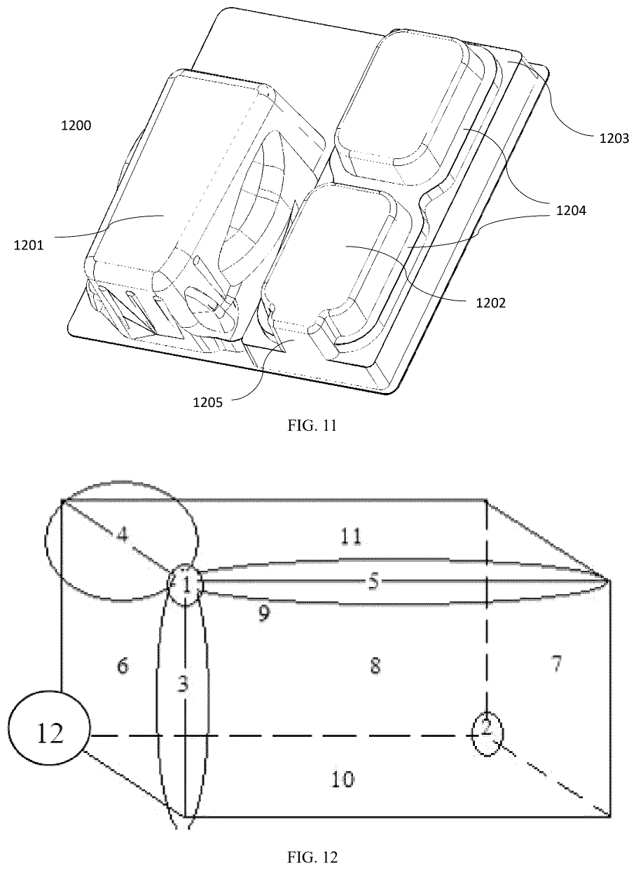

FIG. 12 shows the order of impact locations for a drop test of shipper boxes containing the consumer products described herein.

### EXAMPLES

The consumer product according to an example of the invention was assessed in a drop test to check for premature activation. Each consumer product tested herein comprises cartridges that include a rupture mechanism of the type described in FIG. 1 and U.S. Pat. Nos. 10,561,754, 10,561,755 and 10,561,756. When the cartridges are dropped, the rupture mechanism can prematurely activate, i.e. shocks caused by dropping can cause the rupture elements to pierce the sealing substrate and allow volatile composition to come into contact with the membrane and evaporate.

### Example 1: Drop Testing for Consumer Products

Two types of consumer product were prepared and tested as described below. Each consumer product was dropped 30 times from a height of 1 metre onto a hard floor.

### First Type of Consumer Product

The first type of consumer product included a mixed PET/paperboard blister outer packaging having a paperboard back and moulded PET front. These consumer products included (i) a housing for holding the cartridges, where a cartridge is provided within the housing, and (ii) a sheet-like shock-absorbing insert of the type disclosed in FIGS. 5A and 5B, containing two holes and accommodating two cartridges. While each consumer product contained three cartridges in total, premature activation did not occur for the cartridge stored within the housing, and was only observed for the two cartridges stored outside the housing in the blister.

TABLE 1
Drop test results for first type of consumer product

| | | | No. of |
|---|---|---|---|
| | Shock- | | premature |
| Consumer | absorbing | | activations |
| Product | insert | Shock-absorbing part | from 30 drops |
| A (Comparative) | No | No | 2 |
| B (Comparative) | No | Yes, PET bulge on side | 3 |
| C (Comparative) | No | Yes, two PET bulges on | 0 |
| | | side (rounder than B with | |
| | | greater contact area to | |
| | | reduce impact pressure) | |
| D (Comparative) | No | Yes, PET bulge on base | 2 |
| E (Inventive) | Yes | Yes, PET bulge on base | 0 |

The blister used for Consumer Product E is shown in FIG. 11. The blisters used for the other Consumer Products had corresponding designs but held the cartridges back-to-back rather than using a shock-absorbing insert.

### Second Type of Consumer Product

The second type of consumer product tested included an entirely paperboard outer packaging forming a box. This type included a shock-absorbing insert of the type disclosed in FIG. 6, with either two or four cartridges per box.

TABLE 2
Drop test results for second type of consumer product

| | | | | No. of |
|---|---|---|---|---|
| | | | | premature |
| | | Shock- | Shock- | activations |
| Consumer | No. of | absorbing | absorbing | from 30 |
| Product | Cartridges | insert | portion on base | drops |
| F2 (Comparative) | 2 | No | No | 1 |
| F4 (Comparative) | 4 | No | No | 3 |
| G2 (Comparative) | 2 | No | Yes, paperboard | 2 |
| | | | legs/flaps | |
| G4 (Comparative) | 4 | No | Yes, paperboard | 2 |
| | | | legs/flaps | |
| H2 (Inventive) | 2 | Yes | No | 0 |
| H4 (Inventive) | 4 | Yes | No | 2 |
| I2 (Inventive) | 2 | Yes | Yes, paperboard | 0 |
| | | | legs/flaps | |
| I4 (Inventive) | 4 | Yes | Yes, paperboard | 0 |
| | | | legs/flaps | |

Consumer Products F and G each included an insert corresponding to that disclosed in FIG. 6, except that it did not comprise legs. This meant that the insert was unable to absorb shocks, and forces applied to the base of Consumer Products F and G were transmitted to the cartridges, causing premature activation. Consumer Products H and I included an insert as depicted in FIG. 6. Where the Consumer Products included 4 cartridges, they involved a larger insert comprising four slots, rather than two.

### Example 2: Drop Testing for Shipper Boxes Comprising Multiple Consumer Products

Consumer products C, D, E, F2, G2, I2, and I4 were packaged into shipper boxes of the type that are used for wholesale of consumer products. Two shipper boxes were packed for each product, with 12 consumer products packed in each shipper box.

Drop tests were conducted for these shipper boxes to mimic the forces that the consumer products would be exposed to during the supply chain. Each shipper box was dropped 12 times from a height of 60 cm onto a hard floor. Each drop was performed on a different point/edge/surface, as numbered in FIG. 12. The number of premature activations was recorded.

TABLE 3
Drop test results for shipper boxes packed with consumer products

| Consumer Product | No. of premature activations |
|---|---|
| C (Comparative) | 1 |
| D (Comparative) | 1 |
| E (Inventive)* | 0 |
| F2 (Comparative) | 3 |
| G2 (Comparative) | 2 |
| I2 (Inventive) | 0 |
| I4 (Inventive) | 0 |

*To confirm the benefits of the invention, Consumer Product E was tested with six shipper boxes in total, resulting in 3x the number of drop tests than the corresponding Comparative products and no premature activations.

The drop test results show that the consumer product according to the invention advantageously alleviates the issue of premature activation of refill cartridges for volatile composition dispensers. In particular, the presence of a shock-absorbing insert that supports the peripheral seal regions of cartridges described herein advantageously reduces the incidence of premature activation.

The shipper drop tests also demonstrated that Consumer Product C (which passed the individual drop test of Example 1), resulted in a premature activation under conditions simulating a supply chain. Comparative Product C also resulted in a 73% incidence of marks on the sealing substrate, which marks were caused by impact of the rupture mechanism on the sealing substrate and are indicative of a weakened sealing substrate and pre-failure. In contrast, Consumer Product E had only a 24% incidence of marks on the sealing substrate. These results confirm the advantages provided by the shock-absorbing insert supporting the peripheral seal areas of the cartridges.

As used in this specification and the appended claims, the singular forms “a”, “an”, and “the” include plural references unless the content clearly dictates otherwise. Thus, for example, “a volatile material” may include more than one volatile material. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular configurations of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product comprising:
(i) an outer packaging;
(ii) one or more cartridges packaged within the outer packaging, the one or more cartridges comprising:
a reservoir containing a volatile composition in liquid form and having an opening;
a membrane enclosing the opening of the reservoir, the membrane configured to allow evaporation of the volatile composition;
a sealing substrate enclosing the opening of the reservoir; and
a peripheral seal area in which a peripheral region of the reservoir and membrane are sealed together, and
(iii) a shock-absorbing insert within the outer packaging, wherein the shock-absorbing insert supports the peripheral seal area of the one or more cartridges.

2. The consumer product according to claim 1, wherein the shock-absorbing insert is in direct contact with the peripheral seal area of the one or more cartridges.

3. The consumer product according to claim 1, wherein the peripheral seal area comprises:
an outer peripheral seal area in which a peripheral region of the reservoir and membrane are sealed together; and
an inner peripheral seal area in which a peripheral region of the sealing substrate is sealed to the reservoir,
wherein the shock-absorbing insert is configured to support the outer peripheral seal area and/or the inner peripheral seal area.

4. The consumer product according to claim 1, wherein:
the peripheral seal area comprises one or more secondary reservoir portions between the inner peripheral seal area and the outer peripheral seal area; and
the shock-absorbing insert is configured to support the one or more secondary reservoir portions.

5. The consumer product according to claim 1, wherein the shock-absorbing insert is substantially not in contact with a region of the one or more cartridges other than the peripheral seal area.

6. The consumer product according to claim 1, wherein at least 60% of a contact area between the shock-absorbing insert and the one or more cartridges is between the shock-absorbing insert and the peripheral seal area.

7. The consumer product according to claim 1, wherein:
the one or more cartridges comprise a rupture mechanism located between the membrane and sealing substrate, the rupture mechanism configured to, upon actuation, rupture the sealing substrate; and
a projection of the rupture mechanism onto the membrane in a direction perpendicular to the membrane does not overlap with a contact area between the membrane and the shock-absorbing insert.

8. The consumer product according to claim 7, wherein the rupture mechanism comprises one or more rupture elements configured to rupture the sealing substrate when an actuation pressure is applied to one or more actuation regions on the membrane, and the actuation regions do not overlap with a contact area between the membrane and the shock-absorbing insert.

9. The consumer product according to claim 1, wherein the outer packaging comprises a shock-absorbing portion located at a base region of the outer packaging,
wherein one or more of (A) and (B) applies:
(A) the shock-absorbing portion increases the stability of the consumer product when placed on a flat surface, as measured by determining a minimum angle at which the consumer product topples;
(B) the shock-absorbing portion is formed from a paper-based material (e.g. paperboard) or a plastics material (e.g. polyethylene terephthalate).

10. The consumer product according to claim 1, wherein the shock-absorbing insert comprises:
a first part supporting the peripheral seal area of the one or more cartridges; and
a second part comprising one or more legs or flaps.

11. The consumer product according to claim 1, wherein the shock-absorbing insert comprises:
a sheet having one or more holes, where the reservoirs of the one or more cartridges are received by the one or more holes such that the peripheral seal areas of the one or more cartridges are supported by the sheet; and
one or more legs or flaps extending from the sheet, wherein the one or more legs or flaps are offset from a contact area between the sheet and peripheral seal areas.

12. The consumer product according to claim 1, wherein the shock-absorbing insert comprises:
a base portion;
a pair of intermediate walls extending from opposing edge regions of the base portion, the intermediate walls together comprising one or more pairs of opposing cut-out regions, where each pair of opposing cut-out regions forms a slot within which one of the one or more cartridges is disposed;
a pair of external walls extending from the base portion, the external walls being external to the intermediate walls; and
one or more legs or flaps extending from the base portion.

13. The consumer product according to claim 12, wherein the one or more legs or flaps are offset from a contact area between the base portion and the one or more cartridges.

14. The consumer product according to claim 1, wherein the shock-absorbing insert consists essentially of a paper-based material.

15. The consumer product according to claim 1, wherein the shock-absorbing insert is formed from a single piece of paper-based material.

16. The consumer product according to claim 1, wherein the outer packaging does not contact a part of the one or more cartridges other than the peripheral seal area.

17. The consumer product according to claim 1, wherein the outer packaging does not contact the one or more cartridges.

18. The consumer product according to claim 1, wherein the outer packaging comprises polyethylene terephthalate (PET) and/or paperboard.

19. The consumer product according to claim 1, wherein the shock-absorbing insert is self-supporting when placed outside of the outer packaging.

20. The consumer product according to claim 1, wherein the consumer product comprises two or more cartridges and the two or more cartridges are not in direct contact.
